(19) United States
(12) Patent Application Publication
(10) Pub. No.: US 2005/0158306 A1
(43) Pub. Date: Jul. 21, 2005

(54) Treatment for neuro-degenerative disease by a blood cleansing system using monoclonal antibodies

(76) Inventor: James A. Halikas, Naples, FL (US)

Correspondence Address: James A. Halikas, M.D., 783 Tramore Lane, Naples, FL, 34108, US

(21) Appl. No.: 11/007,875

(22) Filed: Dec. 9, 2004

Related U.S. Application Data
(60) Provisional application No. 60/537,069, filed on Jan. 16, 2004.

(51) Int. Cl. A61K 39/395; C02F 1/44

(52) U.S. Cl. 424/140.1; 210/645

(57) ABSTRACT

Neuro-degenerative diseases such as Alzheimer's Disease with circulating molecules that pass through the Blood Brain Barrier which can generate a monoclonal antibodies specific to the offending circulating molecules are treated with an external blood cleansing system utilizing the monoclonal antibodies to extract the offending molecules.

### CROSS-REFERENCE TO RELATED APPLICATION

This application is a Continuation-In-Part of the Provisional Application No. 60/537,069 filed on Jan. 16, 2004

### STATEMENT REGARDING FED SPONSORED R & D

(none)

### BACKGROUND OF THE INVENTION

Alzheimer's disease is the most common form of progressive dementia primarily affecting individuals over the age of 65. The disease leads to the inevitable destruction of neurons, and ultimately to death within 7 to 10 years. Characteristics of AD are memory impairment, loss of decision-making ability and loss of judgment.

While a definite cause for AD has not yet been precisely delineated, several etiologies have been proposed genetic factors appear to be significant in the development of most cases of AD. Mutations in the amyloid precursor protein (AAP) gene (chromosome 1), the presenilin 1 gene (chromosome 14), and the presenilin 2 gene (chromosome 1) produce an autosomal dominant pattern of inheritance. An abnormal sub unit of APP (beta-amyloid), by various mechanisms related to the mutations, are overproduced. The accumulation of beta-amyloid initiates the cascade of cell death, disruption of cell membranes, inflammatory response, neurofibrillary tangle (NFT) formation, and cerebral amyloid angiopathy. Apolipoprotein (ApoE), a normal protein involved in the metabolism of cholesterol and lipoprotein, has been linked to the development of AD. ApoE exists in three isoforms, one of which (Apo-E4) appears to significantly impact on brain cholinergic activity and/or, by a variety of mechanisms, increases the risk of AD.

AD is also believed to be caused by an amyloid disorder in which soluble proteins fold abnormally into a packed shape which is stable and insoluble. They are found as bundles of insoluble helical fibers within neurons as neuro fibrillary tangles at autopsy. The neuritic plaques of AD are believed to be peptide residues derived from an amyloid precursor protein (AAP). The AD plaques are formed from 40-43 peptide blocks referred to as “A_”. The A_ peptide is made by neurons in the brain but does not cross the blood brain barrier to the body. Other advances have focused on amyloid β (Aβ) peptides. Treating antibodies will reduce the amyloid antigens. Amyloid plaques are found as insoluble deposits between neurons which accumulate in the cortex and hippocampus of the brain. These are composed of amyloid-beta (ABeta) protein fragments: ABeta40 and ABeta42 which are produced from the cleavage of the transmembrane protein beta Amyloid precursor (AAP).

Antibodies to beta-amyloid decrease the movement of blood to brain transfer of beta Amyloid peptide. Active immunization involves immunization with an antigen (ABeta) in order to elicit an immune response, thereby leading to the production of antibodies. Passive immunization involves the injection of antibody (to ABeta) or immune serum into a naive recipient.

Peripheral anti-AB antibodies promote clearance from the brain by altering CNS and plasma AB equilibrium where m266 acts as a “sink” by shifting the equilibrium of AB from the brain to the plasma, the first phase micro-adsorption technique, where AB can then be removed by step two of this technique using the antibody to draw the AB out of the blood. Plasma AB levels increased a thousand fold over control mice that were not injected with m266 antibody. “m266” is a monoclonal antibody that causes a massive increase in the amount of central nervous system-derived amyloid-beta peptide deposited into the plasma. This antibody appears to work by altering the equilibrium of AB levels in the plasma, CSF and brain. Soluble AB levels appear to correlate better with neuro-degeneration. m266 antibody reduces the brain's soluble pool of AB both by increasing brain clearance and sequestering AB in plasma.

Antibodies specific to A_ peptide have been developed in an attempt to bind it and render it harmless. Unfortunately, while the accumulation of the undesirable peptide was reduced, serious complications arose in human subjects which resulted in a termination of a clinical study. Anti-Aβ therapies have also been considered which may use antibodies or Aβ lowering agents. The clinical trials using AB42 as the antigen were halted in phase II due to the CNS inflammation in some Alzheimer patients.

The art described in this section is not intended to constitute an admission that any patent, publication or any other information referred to herein is “prior art” with respect to this invention unless specifically designated as such.

### BRIEF SUMMARY OF THE INVENTION

The invention provides a therapy for neuro-degenerative diseases which involve a circulating molecule that-crosses the blood brain barrier such as is present in Alzheimer's Disease. The invention uses monoclonal antibodies to the circulating molecules that are causing damage in such neuro-degenerative diseases in combination with an external blood device, to be described below, such that the monoclonal antibody is kept away from the patient to avoid any complications. Monoclonal antibodies (MAb) for AD have already been developed and other MAb may be developed and used in connection with Alzheimer's disease or other neuro-degenerative diseases. The MAbs are readily prepared for any molecule that is to tracked and bound to a MAb for removal.

Micro-adsorption is also readily performed using conventional dialysis or adsorption columns which incorporate or bind the specific MAb required for the particular neuro-degenerative disease. Microadsorbtion is but one of the currently available separation technologies encompassed by this proposal. Plasma exchange and dialysis are other existing separating technologies available today. Others may be developed which will utilize this blood cleansing principle. All existing and future separation technologies are meant to be encompassed this invention.

### BRIEF DESCRIPTION OF THE DRAWING

FIG. 1 is a perspective view of a blood cleansing system.

### DETAILED DESCRIPTION OF THE INVENTION

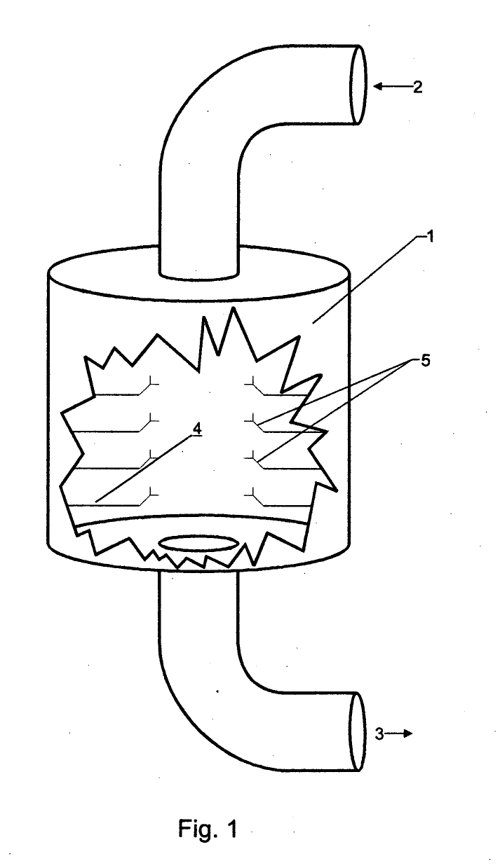

FIG. 1 represents a blood or plasma cleansing system including a micro-adsorption column 1 which is operated externally of a patient's body. The column 1 has a blood or plasma inlet connection 2 coming from the patient and a blood return connection 3 which returns the blood or plasma back to the patient. In the interior of the column 1 there are located ligands 4 by which the antibody 5 will attach via an appropriate bond. The patient's blood or plasma passes through the column 1 and antigens, which are contained in the patient's blood or plasma will attach themselves to the antibody 5. The blood is returned to the patient, minus a certain amount of the antigens.

The treatment of neuro-degenerative disease under the invention involves the use of specific monoclonal antibodies (MAbs) to the circulating compound in the blood which will bind the MAb along with an external blood or plasma cleansing system that ensures that the MAb is not exposed to the brain of the patient. The circulating compounds may be amyloid peptides, prions or any other abnormal molecules which accumulate in the brain that crosses the blood-brain barrier.

The blood cleansing system may be any of the dialysis or micro-adsorbtion technologies currently known, but modified according to FIG. 1, which allow for selective removal of specific compounds from whole blood or plasma including the technologies described by G A Ameer, E A Grovender et al. A novel immunoadsorption device for removing _{—}2-microglobulin from whole blood, Kidney Intl., 59, p. 1544-1550, 2001 or the Prosorba Column already in use.

The monoclonal antibodies of the invention may be produced as needed to be specific to the circulating compound in the blood which crosses the brain-blood barrier. Suitable existing MAbs for Alzheimer Disease include the AB3D6 from elan Pharmaceuticals (San Francisco Calif.) as discussed in a paper by Jhonson-Wood K, Lee M., Motter R., Hu K., Gordon M., Tan H., Games D., Leiderburg I., Schenk D., Suebert P., and McConlogue L. (1977) Amyloid precursor protein processing and Aβ42 deposition in a transgenic mouse model of Alzheimer Disease. Proc. Natl. Acad. Sci. USA 1550-1556; mAbmc1 from DAKO Company (Glostrup, Denmark) and Eli Lilly and Company's M266 monoclonal antibody.

A patient in need of therapy would be attached to the blood cleansing system with the required monoclonal antibodies included. The blood or plasma circulation would be run as directed by a physician. Typically, the initial session would be of longer duration and at a higher frequency until the undesirable circulating compound's level has decreased sufficiently to either reverse the effects of the disease or until stabilization of the progression of the disease occurred.

Any removal of the circulating compound may at least slow down the progression of the disease, performing the treatments more frequently and for longer periods of time could increase the therapeutic effect.

1. A treatment for Alzheimer's Disease including the steps of attaching a patient believed to have Alzheimer's Disease to a treatment of an external blood cleansing system, utilizing monoclonal antibodies specific to amyloid peptides, periodically running said treatment until sufficient amyloid peptide is extracted from the brain and the blood to a level stabilizing and improving the clinical condition of the patient.

2. The treatment of claim 1 including the steps of repeated treatments on an ongoing basis to remove later productions of the amyloid peptides by the patients body.

3. A treatment for any neuro-degenerative disease in a patient in which abnormal molecules are manufactured which accumulate in the brain of said patient, including the steps of subjecting said patient to a treatment of an external blood cleansing system and employing monoclonal antibodies specific to said abnormal molecules and periodically running said treatment until sufficient abnormal molecules are extracted from the brain and the blood until a level of stabilization and improvement of the clinical condition of the patients has been achieved.

4. The treatment of claim 3 including the steps of repeating said blood cleansing treatment on an ongoing basis to remove later productions of said abnormal molecules by the patient's body.

5. A treatment for a neuro-degenerative disease in a patient caused by prions which accumulate in the brain of said patient including the steps of subjecting said patient to an external blood cleansing treatment which treatment employs monoclonal antibodies specific to said prions and periodically running said blood cleansing treatment until sufficient prions are extracted from said brain and said blood to a level of stabilizing and improving the clinical condition of said patient.
